(19) United States
(12) Patent Application Publication
(10) Pub. No.: US 2012/0264910 A1
(43) Pub. Date: Oct. 18, 2012

(54) PROCESS FOR COUPLING EPOXIDES AND CARBON DIOXIDE

(75) Inventors: Peter Deglmann, Mannheim (DE); Anna Katharina Brym, Limburgerhof (DE); Joachim Dengler, Tacherting (DE); Petra Lutz-Kahler, Weiterstadt (DE); Volker Warzelhan, Weisenheim am Berg (DE); Bernhard Rieger, Munchen (DE); Stephan Klaus, Munchen (DE); Maximilian Lehenmeier, Munchen (DE)

(73) Assignee: BASF SE, Ludwigshafen (DE)

(21) Appl. No.: 13/445,251

(22) Filed: Apr. 12, 2012

Related U.S. Application Data
(60) Provisional application No. 61/474,761, filed on Apr. 13, 2011.

(30) Foreign Application Priority Data
Apr. 13, 2011 (EP) 11162227.0

(51) Int. Cl. C08G 67/00; C07D 317/36; C07F 15/02

(52) U.S. Cl. 528/405; 546/12; 548/402; 549/230

(57) ABSTRACT

The present invention relates to a process for preparing carbonates by reacting propylene oxide, ethylene oxide, styrene oxide and/or cyclohexene oxide with carbon dioxide in the presence of one or more catalysts of the formula I

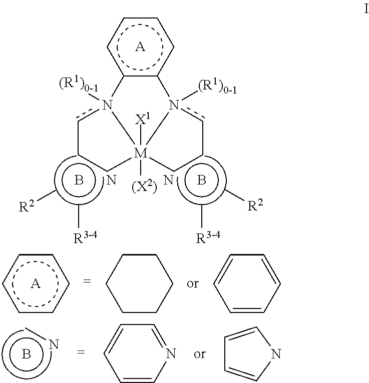

where
R¹ is hydrogen, C₁-C₆-alkyl, C₁-C₆-haloalkyl, NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano;
R³, R⁴ are each hydrogen or together are a butadienylene moiety which bears the R⁵ substituent;
R⁵ is C₁-C₄-alkyl, C₁-C₄-haloalkyl, halogen, amino, nitro, C₁-C₆-alkoxy or cyano;
M is Zn(II), Mg(II), Cr(II), Cr(III), Co(II), Co(III), Fe(II) or Fe(III); and
X¹, X² are each OCOCH₃, OCOCF₃, OSO₂C₇H₇ or halogen.

More particularly, the invention relates to a process for preparing cyclic carbonates, and to a process for preparing aliphatic polycarbonates using these catalysts I, and to particularly preferred catalysts of the formulae Ia and Ib.

The present invention relates to a process for preparing carbonates by reacting propylene oxide, ethylene oxide, styrene oxide and/or cyclohexene oxide with carbon dioxide in the presence of one or more catalysts of the formula I

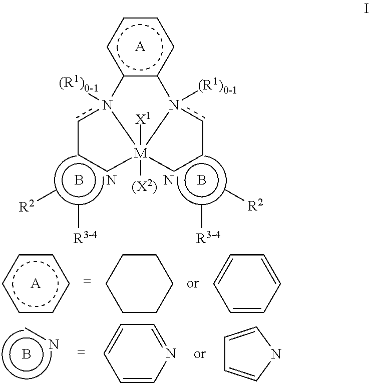

where
R¹ is hydrogen, C₁-C₆-alkyl, C₁-C₆-haloalkyl, NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano; R³, R⁴ are each hydrogen or together are a butadienylene moiety which bears the R⁵ substituent;
R⁵ is C₁-C₄-alkyl, C₁-C₄-haloalkyl, halogen, amino, nitro, C₁-C₆-alkoxy or cyano;
M is Zn(II), Mg(II), Cr(II), Cr(III), Co(II), Co(III), Fe(II) or Fe(III); and
X¹, X² are each OCOCH₃, OCOCF₃, OSO₂C₇H₇ or halogen.

More particularly, the invention relates to a process for preparing cyclic carbonates, and to a process for preparing aliphatic polycarbonates using these catalysts I, and to particularly preferred catalysts of the formulae Ia and Ib.

Catalysts of the formula I which do not have further substitution in the pyridine ring have been described in DE 101 30 220 for the polymerization of olefins. This document does not make any mention of possible suitability of these catalysts for the coupling of epoxides and carbon dioxide.

In addition, Eur. J. Inorg. Chem. 2011, p. 336 to 343 mentions catalysts of the formula Ic which do not bear any further substituents in the quinoline ring. These catalysts are used for preparation of cyclic propylene carbonate. However, no statement is made regarding the efficiency (turnover frequency, TOF) of these catalysts.

It was an object of the present invention to provide efficient catalysts for an improved process for preparing cyclic carbonates and especially polycarbonates by coupling of epoxides and carbon dioxide.

Surprisingly, this object is efficiently achieved by the catalysts of the formula I mentioned at the outset.

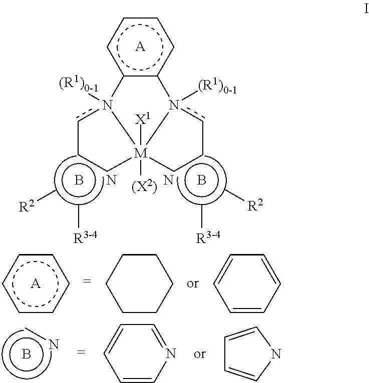

where
R¹ is hydrogen, C₁-C₆-alkyl, C₁-C₆-haloalkyl, NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano;
R³, R⁴ are each hydrogen or together are a butadienylene moiety which bears the R⁵ substituent;
R⁵ is C₁-C₄-alkyl, C₁-C₄-haloalkyl, halogen, amino, nitro, C₁-C₆-alkoxy or cyano;
M is Zn(II), Mg(II), Cr(II), Cr(III), Co(II), Co(III), Fe(II) or Fe(III); and
X¹, X² are each OCOCH₃, OCOCF₃, OSO₂C₇H₇ or halogen.

The catalysts are described in detail hereinafter:

The ring A may be a saturated 1,2-cyclohexylene diradical and an unsaturated 1,2-phenylene diradical. The 1,2-cyclohexylene diradical is preferably disubstituted in the trans diequatorial positions.

The B ring is a quinoline or preferably a pyrrole or pyridine ring, where the B ring is preferably substituted by the R² or R⁵ radicals.

The dotted line C . . . N means a single bond or no bond.

The substituent R¹ is hydrogen, C₁-C₆-alkyl, C₁-C₆-haloalkyl, NR′₄—(CH₂)₂₋₆, where R′ is C₁-C₆-alkyl, and is especially hydrogen or NR′₄—(CH₂)₂₋₆, where R′ is C₁-C₄-alkyl.

The substituent R² may be at any position on the B ring and is hydrogen, C₁-C₄-alkyl, C₁-C₄-haloalkyl, halogen, amino, nitro, C₁-C₆-alkoxy or cyano, and especially methyl, tert-butyl, trifluoromethyl, fluorine, chlorine, bromine, iodine, methoxy, trifluoromethoxy or cyano. More preferably, the electron-withdrawing substituents are: trifluoromethyl, fluorine, chlorine, trifluoromethoxy or cyano. The ring B may bear one or two substituents R².

C₁-C₄-Alkyl is methyl, ethyl, n-propyl, isopropyl, n-butyl, isobutyl, sec-butyl, tert-butyl.

C₁-C₆-Alkyl comprises the aforementioned definitions of C₁-C₄-alkyl, and also n-pentyl, 1,1-dimethylpropyl, 1,2-dimethylpropyl, 2,2-dimethylpropyl, 1-ethylpropyl, n-hexyl, 1-ethyl-2-methylpropyl, 1,1,2-trimethylpropyl, 1-ethylbutyl, 1-methylbutyl, 2-methylbutyl, 1,1-dimethylbutyl, 1,2-dimethylbutyl, 2,2-dimethylbutyl, 1,3-dimethylbutyl, 2,3-dimethylbutyl, 2-ethylbutyl, 2-methylpentyl and 3-methylpentyl.

C₁-C₄-Haloalkyl is a C₁-C₄-alkyl group which bears preferably 1, 2 or 3 halogen atoms, especially fluoromethyl, difluoromethyl, trifluoromethyl, fluoroethyl, difluoroethyl, trifluoroethyl, chloromethyl, bromomethyl or iodomethyl.

The substituents R³ and R⁴ are each hydrogen or together are a butadienylene moiety which forms a quinoline ring with the pyridine ring B or an indole ring with the pyrrole ring B. The butadienylene moiety bears one substituent R⁵, which is C₁-C₄-alkyl, C₁-C₄-haloalkyl, halogen, amino, nitro, C₁-C₆-alkoxy or cyano, and especially methyl, tert-butyl, trifluoromethyl, fluorine, chlorine, bromine, iodine, methoxy, trifluoromethoxy or cyano. Preferably, R³ and R⁴ are each hydrogen.

The central atom M is a Zn(II), Mg(II), Cr(II), Cr(III), Co(II), Co(III), Fe(II) or Fe(III) atom, preferably a Zn(II), Co(II), Co(III), Fe(II) or Fe(III) atom, and especially preferably an Fe(II) or Fe(III) atom.

The X¹ and X² ligands are the monoanions: OCOCH₃ (acetate), OCOCF₃ (trifluoroacetate), OSO₂C₇H₇ (benzylsulfonate), or halide (fluoride, chloride, bromide, iodide), preferably OCOCH₃ (acetate), OCOCF₃ (trifluoroacetate) or halide, and especially preferably OCOCH₃ (acetate) or chloride.

Preference is given to catalysts of the formula Ia (pyridine ligand),

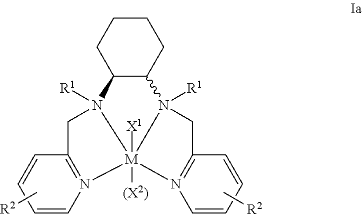

where
R¹ is hydrogen, C₁-C₆-alkyl, C₁-C₆-haloalkyl, NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano; the R² radical is preferably in the free ortho position and/or the para position on the pyridine ring; preferably mono- or disubstitution is present;
M is Zn(II), Co(II), Co(III), Fe(II) or Fe(III); and
X¹, X² are each OCOCH₃, OCOCF₃, OSO₂C₇H₇ or halogen;
   of the formula Ib (pyrrole ligand),

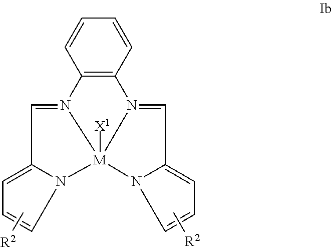

where
R² is hydrogen, C₁-C₄-alkyl, C₁-C₄-haloalkyl, halogen, amino, nitro, C₁-C₆-alkoxy or cyano; the R² radical is preferably in the 3 and/or 5 position on the pyrrole ring; preferably mono- or disubstitution is present;
M is Zn(II), Co(II), Co(III), Fe(II) or Fe(III); and
X¹ is OCOCH₃, OCOCF₃, OSO₂C₇H₇ or halogen.

The inventive catalysts are suitable for coupling carbon dioxide and epoxides to give cyclic carbonates or polyalkylene carbonates. Epoxides are understood to mean propylene oxide, ethylene oxide, styrene oxide and cyclohexene oxide. Preference is given to propylene oxide and cyclohexene oxide, and it is also possible to use these as a mixture in the reaction.

In some cases, mixtures of cyclic carbonates or polyalkylene carbonates are obtained. In these cases, the ratio of the cyclic carbonates to the polyalkylene carbonates can be shifted by establishing suitable process parameters (pressure, temperature, stirrer speed, reaction time, proportion of water, and especially cocatalysts) and selecting catalysts suitable for the polymerization. Useful cocatalysts include bis(triphenylphosphine)iminium chloride, tetra-n-butylammonium bromide, 1-methylimidazole and 4-(dimethylamino)pyridine; among these, preference is given to bis(triphenylphosphine)iminium chloride or tetra-n-butylammonium bromide.

For the polymerization to give polyalkylene carbonates, especially the catalysts of the formula Ia have been found to be advantageous.

In addition, for this purpose, the incorporation of Lewis acid groups, for example trifluoromethyl or cyano groups, into the ring B has been found to be advantageous.

In addition, it has been found to be advantageous for this purpose to incorporate anchor groups which can coordinate an anionic polymer chain end and thus prevent “backbiting”. This backbiting describes a reaction in which a cyclic carbonate is eliminated as a result of intramolecular attack of the nucleophilic polymer chain end. In general, it is assumed that coordinating anchor groups stabilize the polymer chain ends and thus prevent backbiting. A suitable anchor group has been found to be the substituents R¹ defined as N⁺R′₄—(CH₂)₂₋₆, where R′ is C₁-C₆-alkyl. In addition, the polymerization proceeds more rapidly as a result of the incorporation of such anchor groups.

The preferred inventive iron or cobalt catalysts have the following advantages:

A nontoxic metal, especially iron (or cobalt), is used as a catalytically active site for the coupling of carbon dioxide (CO₂) with epoxides. The workup of the carbonates formed, especially of the polyalkylene carbonates formed, is found to be simpler since there is no need to remove the catalyst quantitatively.

In the range of 40-120° C. (preferably 80-100° C.) and CO₂ pressures of 1-50 bar (preferably 15-30 bar), the inventive iron catalysts are suitable for preparation of cyclic carbonate—especially cyclic propylene carbonate.

The inventive iron catalysts afford alternating copolymers in the reaction of CO₂ and cyclohexene oxide (CHO).

The proportion of cyclic cyclohexylene carbonate as a by-product can be adjusted via pressure and temperature.

Better handling of the inventive catalyst compared to the heterogeneous zinc glutarate described in WO 2003/029325.

Better handling of the inventive catalyst which, compared to the cobalt catalyst with salen ligands described in WO 2008/136591, is not as moisture- and oxygen-sensitive and can be synthesized in a simpler manner.

### Preparation of the Ligands

The ligands are generally synthesized by the route described hereinafter for the ligand a).

### a) (1S,2S)—N,N′-bis(Pyridin-2-ylmethylene)cyclohexane-1,2-diimine

To a solution of 1.91 ml of pyridine-2-carboxaldehyde (20.0 mmol, 2.0 equiv.) in 40 ml of ethanol were added 1.09 g of (S,S)-cyclohexanediamine (9.5 mmol, 1.0 equiv.). The reaction mixture was stirred at room temperature (RT) for 20 h. Subsequently, the solvent was removed under reduced pressure and the product was recrystallized from CH₂Cl₂/Et₂O. Yield: 1.8 g of yellow powder (6.16 mmol, 65%).

¹H NMR (400 MHz, CDCl₃, 24° C.): δ (ppm) 8.54 (dd, J=4.9, 1.3 Hz, 2H; py-H), 8.30 (s, 2H; N═C—H), 7.87 (d, J=7.7 Hz, 2H; py-H), 7.63 (dd, J=7.7, 1.3 Hz, 2H; py-H), 7.21 (ddd, J=7.7, 4.9, 1.3 Hz, 2H; py-H), 3.53 (m, 2H, *C—H), 1.85 (m, 6H; cyclohex), 1.51 (d, 2H; cyclohex). ESI-MS (m/z) calculated for C₁₈H₂₀N₄: 292.38; found 293.1 (M⁺), 315.1 (M⁺+Na).

### b) (1S,2S)—N,N′-bis(Pyridin-2-ylmethyl)cyclohexane-1,2-diamine

1.20 g of (1S,2S)—N,N′-bis(pyridin-2-ylmethylene)cyclohexane-1,2-diamine (ligand a) (4.1 mmol, 1.0 equiv.) were added to a solution consisting of 1.54 g of sodium borohydride NaBH₄ (41.0 mmol, 10 equiv.) in 60 ml of methanol at 0° C. The reaction solution was stirred at RT for 2 days. Subsequently, 5 ml of water were added and the solution was extracted with 3×75 ml of CH₂Cl₂ and finally with 3×50 ml of water. The organic phases were dried over Na₂SO₄ and the solvent was removed under reduced pressure.

Yield: 1.21 g of brown powder (4.1 mmol, 99%).

¹H NMR (400 MHz, CDCl₃, 24° C.): δ (ppm) 8.44 (s, 2H; py-H), 7.53 (s, 2H; py-H), 7.33 (s, 2H; py-H), 7.04 (s, 2H; py-H), 3.95 (d, J=14.2 Hz, 2H; N—C—H), 3.75 (d, J=14.2 Hz, 2H; N—C—H), 2.50 (s, 2H; N—H), 2.25 (m, J=5.7 Hz, 2H; cyclohex), 2.06 (d, J=11.2 Hz, 2H; cyclohex), 1.63 (d, J=5.0 Hz, 2H; cyclohex), 1.10 (m, 2H; cyclohex), 1.01 (s, 2H; cyclohex). ¹³C NMR (400 MHz, CDCl3, 24° C.): δ (ppm) 160.69, 149.03, 136.37, 122.28, 121.73, 61.33, 52.50, 31.56, 24.97. ESI-MS (m/z) calculated for C₁₈H₂₄N₄: 297.38; found 297.3 (M⁺), 319.2 (M⁺+Na).

### c) (1S,2S)—N,N′-bis(3-(Trifluoromethyl)pyridin-2-yl)methylene)cyclohexane-1,2-diimine

0.70 ml of 3-(trifluoromethyl)pyridine-2-carboxaldehyde (5.5 mmol, 2.0 equiv.) was dissolved in 30 ml of ethanol, and 0.29 g (2.6 mmol, 2.0 equiv.) of 1,2-(S,S)-diaminocyclohexane was added. The orange solution was stirred at RT for 3 days (reaction was monitored by means of ¹H NMR). The solvent was removed under reduced pressure and the product was obtained as an orange oil. Recrystallization was effected from CH₂Cl₂/Et₂O.

Yield: 0.99 g (or 0.46 g) of yellow-orange powder (2.3 mmol, 93%).

¹H NMR (400 MHz, CDCl₃, 24° C.): δ (ppm) 8.85 (d, J=3.7 Hz, 2H), 8.56 (d, J=1.8 Hz, 2H), 7.90 (d, J=8.0 Hz, 2H), 7.35 (d, J=8.0 Hz, 2H), 3.71 (m, 2H), 2.15 (m, 2H), 1.88 (d, J=8.2 Hz, 4H), 1.55 (m, 2H).

ESI-MS (m/z) calculated for C₂₀H₁₈F₆N₄: 428.37; found 429.1 (M+), 451.1 (M⁺+Na).

### d) (1S,2S)—N,N′-bis(3-(Trifluoromethyl)pyridin-2-yl)methyl)cyclohexane-1,2-diamine

0.30 g of (1S,2S)—N,N′-bis(3-(trifluoromethyl)pyridin-2-yl)methylene)cyclohexane-1,2-diamine (ligand c) (0.7 mmol, 1.0 equiv.) was suspended in 20 ml of dry MeOH and cooled to 0° C. 0.26 g of sodium borohydride (7.0 mmol, 10 equiv.) was added gradually while stirring. Subsequently, the reaction solution was stirred at RT for 12 h. The pale brown solution was filtered and washed with water and dichloromethane, and the solvent was removed under reduced pressure.

Yield: 0.21 g of brown oil (0.49 mmol, 69%).

¹H NMR (400 MHz, CDCl₃, 24° C.): δ (ppm) 8.71 (d, J=4.9 Hz, 2H), 7.89 (d, J=7.6 Hz, 2H), 7.26 (dd, J=7.6, 4.9 Hz, 2H), 4.15 (d, J=15.0 Hz, 2H), 4.02 (d, J=15.0 Hz, 2H), 2.82 (s, 2H), 2.36 (d, J=7.0 Hz, 2H), 2.09 (d, J=12.5 Hz, 2H), 1.69 (m, 2H), 1.22 (dd, J=12.5, 7.0 Hz, 2H), 1.10 (m, 2H). ¹³C NMR (400 MHz, CDCl₃, 24° C.): δ (ppm) 158.63, 151.91, 133.88, 122.14, 121.30, 61.39, 49.05, 31.71, 25.02.

ESI-MS (m/z) calculated for C₂₀H₂₂F₆N₄: 433.1; found 433.1 (M+), 455.1 (M⁺+Na).

### e) (1S,2S)—N,N′-bis(6-Methylpyridin-2-yl)methylene)cyclohexane-1,2-diimine

4.5 g of 6-methylpyridine-2-carboxaldehyde (37.0 mmol, 2.0 equiv.) were dissolved in 50 ml of ethanol, and 2.12 g of 1,2-(S,S)-diaminocyclohexane (18 mmol, 1.0 equiv.) were added. The orange solution was stirred at RT for 12 h. The solvent was removed under reduced pressure and the product was present in the form of an orange oil which was recrystallized from CH₂Cl₂/Et₂O.

Yield: 4.8 [g] of yellow-orange powder (14.98 mmol, 83%).

¹H NMR (400 MHz, CDCl₃, 24° C.): δ (ppm) 8.27 (s, 2H), 7.69 (d, J=7.7 Hz, 2H), 7.48 (t, J=7.7 Hz, 2H), 7.04 (d, J=7.7 Hz, 2H), 3.47 (m, 2H), 2.48 (s, 6H), 1.79 (m, 6H), 1.45 (m, 2H). ¹³C NMR (400 MHz, CDCl₃, 24° C.): δ (ppm) 161.84, 157.90, 154.33, 136.76, 124.22, 118.40, 73.74, 32.84, 24.50, 24.41.

ESI-MS (m/z) calculated for C₂₀H₂₄N₄: 320.43; found 321.3 (M+), 343.1 (M⁺+Na).

### f) (1S,2S)—N,N′-bis(6-Methylpyridin-2-yl)methyl)cyclohexane-1,2-diamine

2 g of (1S,2S)—N,N′-bis(6-methylpyridin-2-yl)methylene)cyclohexane-1,2-diamine (ligand e) (6.24 mmol, 1.0 equiv.) were suspended in 80 ml of MeOH (dry) and cooled to 0° C. 2.34 g of sodium borohydride (62.4 mmol, 10 equiv.) were slowly added dropwise and the mixture was stirred at RT for 12 h. The pale yellow solution was filtered and washed with water and CH₂Cl₂, and the solvent was removed under reduced pressure.

Yield: 1.35 g of yellow powder (4.17 mmol, 67%).

¹H NMR (400 MHz, CDCl₃, 24° C.): δ (ppm) 7.50 (t, J=7.6 Hz, 2H), 7.23 (m, 2H), 6.99 (d, J=7.6 Hz, 2H), 4.00 (d, J=14.2 Hz, 2H), 3.79 (d, J=14.2 Hz, 2H), 2.51 (s, 6H), 2.34 (b, 2H), 2.30 (m, 2H), 2.15 (d, J=13.0 Hz, 2H), 1.71 (d, J=7.1 Hz, 2H), 1.23 (dd, J=13.0, 7.1 Hz, 2H), 1.07 (m, 2H). ¹³C NMR (400 MHz, CDCl₃, 24° C.): δ (ppm) 160.17, 157.72, 136.74, 121.36, 119.24, 61.52, 52.65, 31.77, 25.14, 24.59.

ESI-MS (m/z) calculated for C₂₀H₂₈N₄: 324.46; found 325.3 (M⁺), 347.2 (M⁺+Na).

Ligands with anchor groups (R¹ different than hydrogen)

### g) (1S,2S)—N,N′-Dipropyl-N,N′-bis(pyridin-2-ylmethyl)cyclohexane-1,2-diamine

In a 50 ml Schlenk flask under protective gas, 0.594 g of (S,S)—N,N′-bis(pyridin-2-ylmethyl)-cyclohexane-1,2-diamine (ligand b) (2.0 mmol, 1.0 equiv.) was dissolved in 10 ml of NEt₃. Subsequently, 0.39 ml of iodopropane (0.679 g, 4.0 mmol, 2.0 equiv.) was added. The reaction solution was stirred at 90° C. for 16 h. The volatile components were removed under reduced pressure and the product was washed with 20 ml of H₂O. The solution was extracted three times with 75 ml each time of ethyl acetate and three times with 25 ml each time of water. The collected organic phases were dried over Na₂SO₄ and the solvent was drawn off.

¹H NMR: The spectrum corresponds to that of ligand b. Instead of the N—H singlets of the secondary amines at 2.5 ppm, signals of the methylene group of the anchor spacers have appeared (4H triplet at 2.5 ppm, a 4H sextet at 1.6 ppm and a 6H triplet at 0.9 ppm).

### h) (1S,2S)—N,N′-bis(3-Chloropropyl)-N,N′-bis(pyridin-2-ylmethyl)cyclohexane-1,2-diamine

A 50 ml round-bottom flask was initially charged with 0.70 ml of 3-chloro-1-iodopropane (1.22 g, 6.0 mmol, 2.0 equiv.), and a solution of 0.889 g of (S,S)—N,N′-bis(quinoline-2-methylene)-cyclohexane-1,2-diamine (ligand b) (3.0 mmol, 1.0 equiv.) in 10 ml of NEt₃ was added. A further 10 ml of NEt₃ were added and the mixture was stirred under reflux at 90° C. for 16 hours. The solvent was drawn off, 20 ml of water were added and the mixture was stirred for another 1 h. The reaction solution was extracted with 3×75 ml of ethyl acetate and with 3×25 ml of water.

The product was dried over Na₂SO₄ and the solvent was removed.

Yield: 0.496 g as a brown oil (yield: 37%).

¹H NMR: The spectrum corresponds to that of compound b. Instead of the N—H singlets of the secondary amines at 2.5 ppm, signals of the methylene group of the anchor spacers have appeared (4H triplet at 2.5 ppm, a 4H sextet at 1.6 ppm and a 6H triplet at 0.9 ppm, and a low field-shifted 2H triplet at 3.7 ppm for the terminal halogenated methyl group).

### i) (1S,2S)—N,N′-bis(3-Iodopropyl)-N,N′-bis(pyridin-2-ylmethyl)cyclohexane-1,2-diamine

To a solution of 0.496 g of ligand (h) (1.13 mmol, 1.0 equiv) in 20 ml of acetone were added 1.5 g of sodium iodide (10.0 mmol, 10 equiv.). The reaction solution was stirred at 50° C. under reflux for 20 h. The solvent was removed under reduced pressure.

Yield: 0.154 g as a brown oil (20%).

¹H NMR: The spectrum corresponds to that of compound b. Instead of the N—H singlets of the secondary amines at 2.5 ppm, signals of the methylene group of the anchor spacers have appeared (4H triplet at 2.5 ppm, a 4H quintet at 2.0 ppm and a low field-shifted 2H triplet at 3.2 ppm for the terminal halogenated methyl group).

### j) N,N′-((1S,2S)-Cyclohexane-1,2-diylbis((pyridin-2-ylmethyl)azanediyl))bis(propane-3,1-diyl))-bis(N,N′-dibutylbutane-1-aminium) iodide

In a 100 ml round-bottom flask, 0.45 ml of NBu₃ (0.37 g, 2.0 mmol, 10 equiv.) was added to a solution of 0.126 g of (ligand i) (0.2 mmol, 1.0 equiv.) dissolved in 20 ml of CH₃CN. The reaction solution was stirred at 90° C. under reflux for 48 h. After cooling to RT, the solvent was removed and the yellow oil formed was taken up in diethyl ether and filtered.

Yield: 0.056 g of (ligand k) in the form of dark crystals (28%). The NMR spectra and ESI-MS experiments indicate a mixture of mono- and disubstituted ligands b). ¹H NMR: The spectrum corresponds to that of ligand b). Instead of the N—H singlets of the secondary amines at 2.5 ppm, signals of the methylene group of the anchor spacers have appeared (4H triplet at 2.5 ppm, a 4H quintet at 1.4 ppm and a low field-shifted 2H triplet at 1.3 ppm. For the tributylammonium, the terminal methyl groups are found as an 18H triplet at 0.9 ppm and a 36H multiplet of the spacer group at 1.2-1.4 ppm.

### k) N,N′-((1S,2S)-Cyclohexane-1,2-diylbis((pyridin-2-ylmethyl)azanediyl))bis(propane-3,1-diyl))-bis(N,N′-dibutylbutane-1-aminium) tetrafluoroborate

0.042 g (0.2 mmol, 1.0 equiv) was initially charged in a 50 ml Schlenk flask and dissolved in 7 ml of EtOH (abs.). The flask was wrapped with aluminum foil and 0.016 g of AgBF4 were added in the dark. The reaction solution was stirred at room temperature for 20 h.

Yield: dark brown solid (ligand l). The NMR spectra and ESI-MS experiments indicate a mixture of mono- and disubstituted ligands b). ¹H NMR: The spectrum corresponds to that of ligand b). Instead of the N—H singlets of the secondary amines at 2.5 ppm, signals of the methylene group of the anchor spacers have appeared (4H triplet at 2.5 ppm, a 4H quintet at 1.4 ppm and a low field-shifted 2H triplet at 1.3 ppm). For the tributylammonium, the terminal methyl groups are found as an 18H triplet at 0.9 ppm, and the 24H multiplet of the spacer group at 1.2-1.4 ppm.

### l) N,N′-Di(3,5-dimethylpyrrole-2-methylene)-1,2-cyclohexyldiimine

1.00 g (8.1 mmol) of pyrrole-2-carboxaldehyde was dissolved in 40 ml of ethanol, and 0.439 g (4.05 mmol) of 1,2-(S,S)-diaminocyclohexane was added. The orange solution was stirred at room temperature for 12 hours and the solvent was drawn off under reduced pressure. The product was recrystallized in DCM/ether in order to obtain 0.58 g of a yellow powder (1.82 mmol, 23%).

### Complex Synthesis

The general synthesis of the complexes is effected as described for example 1.

### EXAMPLE 1

### FeCl₂(C₁₈H₂₂N₄)

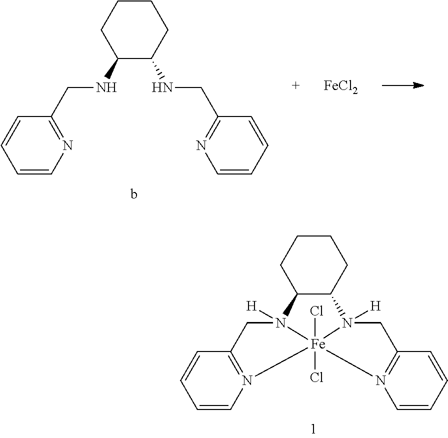

A 100 ml round-bottom flask was initially charged with 1.037 g of ligand b (3.5 mmol, 1.0 equiv.) and 2.21 g of FeCl₂ (17.5 mmol, 5.0 equiv.), and 40 ml of absoluted CH₂Cl₂ were added. The yellow reaction solution was stirred at RT for 20 h. The orange reaction solution was filtered through a syringe filter and the solvent was drawn off under reduced pressure.

¹H/¹³C NMR: NMR spectra are not possible due to the paramagnetism of Fe(II).

FT-IR (cm⁻¹): 3206.24, 2920.64, 2851.37, 1606.49, 1570.51, 1444.51, 1375.98, 1259.92, 1155.67, 1100.36, 1054.23, 1022.38, 763.96, 729.42, 643.86.

FAB-MS (m/z) calculated for C₁₈H₂₂Cl₂FeN₄: 421.15.

### EXAMPLE 2

### (Not Inventive) FeCl₂(C₁₈H₂₀N₄)

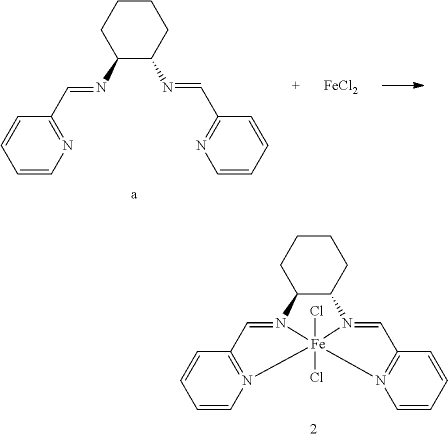

To a solution of 0.5 g (1.69 mmol, 1.0 equiv) of ligand a in 20 ml of CH₂Cl₂ was added 1.0 g (8.35 mmol, 5.0 equiv.) of FeCl₂. After stirring for 24 h, the greenish solution was filtered under a protective gas atmosphere and the solvent was removed under reduced pressure.

Yield: 0.2 g of dark brown crystals (0.48 mmol, 28%).

¹H/¹³C-NMR: NMR spectra are not possible due to the paramagnetism of Fe(II).

FT-IR (cm⁻¹): 2928.81, 2856.96, 2361.11, 2338.39, 1647.34, 1596.21, 1444.53, 1302.80, 1223.41, 1155.76, 1105.30, 1050.08, 1017.22, 821.04, 769.66, 731.05, 697.29, 640.94.

FAB-MS (m/z) calculated for C₁₈H₂₀Cl₂FeN₄: 419.13.

### EXAMPLE 3

### FeCl₂(C₂₀H₂₀F₆N₄)

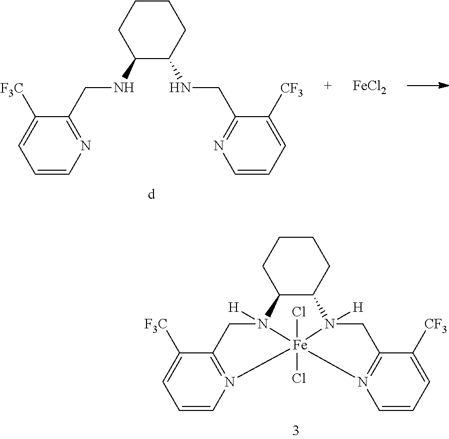

To a solution of 0.1 g of ligand d (0.23 mmol, 1.0 equiv.) in 10 ml of CH₂Cl₂ was added 0.3 g of FeCl₂ (2.5 mmol, 10.0 equiv.). After stirring for 24 hours, the solution was filtered under a protective gas atmosphere, the solvent was removed under reduced pressure and the product was dried therein.

Yield: 0.2 g of brown crystals (0.48 mmol, 28%).

¹H/¹³C-NMR: NMR spectra were not possible due to the paramagnetism of Fe(II).

FT-IR (cm⁻¹): 3214.20, 2929.09, 2860.25, 1597.04, 1449.51, 1372.92, 1318.63, 1257.49, 1170.28, 1125.22, 1086.42, 1049.26, 948.86, 815.52, 729.57, 680.08, 657.11.

FAB-MS (m/z) calculated for C₂₀H₂₀Cl₂F₆FeN₄: 557.14

### EXAMPLE 4

### FeCl₂(C₂₀H₂₆N₄)

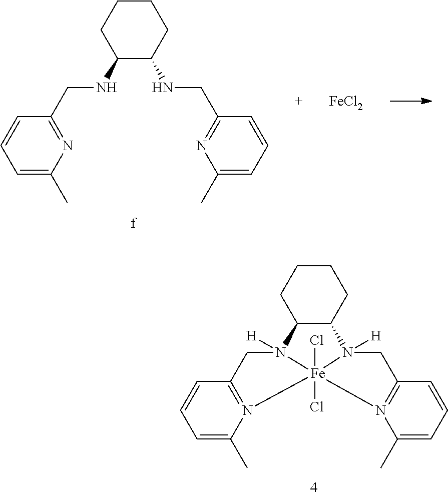

To a solution of 0.15 g of ligand f) (0.46 mmol, 1.0 equiv.) in 10 ml of CH₂Cl₂ was added 0.6 g of FeCl₂ (5.00 mmol, 10.0 equiv.). After stirring for 24 hours, the brown-red solution was filtered under protective gas and the solvent was removed.

Yield: 0.2 g of red-brown crystals (0.29 mmol, 63%).

¹H/¹³C-NMR: NMR spectra were not possible due to the paramagnetism of Fe(II).

FT-IR (cm⁻¹): 2922.38, 2853.05, 1605.54, 1576.40, 1462.57, 1377.08, 1260.09, 1166.03, 1090.34, 1012.87, 791.11, 661.94.

FAB-MS (m/z) calculated for C₂₀H₂₆Cl₂FeN₄: 449.20.

### COMPARATIVE EXAMPLE 5

### FeCl₂(C₂₆H₂₆N₄)

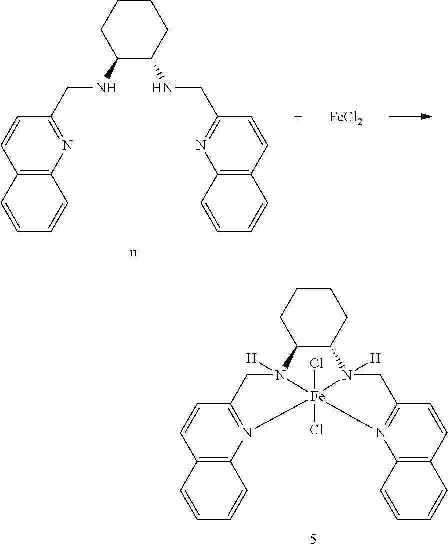

To a solution of 0.25 g of ligand n (prepared according to Eur. J. Inorg. Chem, 2011, p. 336-343) (0.63 mmol, 1.0 equiv.) in 30 ml of CH₂Cl₂ was added 0.4 g of FeCl₂ (1.26 mmol, 5.0 equiv.). After stirring for 24 hours, the red solution was filtered under protective gas and the solvent was removed.

Yield: 0.2 g of dark brown crystals (0.38 mmol, 61%).

¹H/¹³C-NMR: NMR spectra were not possible due to the paramagnetism of Fe(II).

FT-IR (cm⁻¹): 2922.35, 2852.02, 2360.92, 2339.01, 1599.03, 1509.32, 1432.09, 1377.41, 1303.07, 1261.54, 1208.41, 1143.96, 1094.17, 1022.15, 958.52, 870.36, 826.97, 781.21, 748.55.

FAB-MS (m/z) calculated for C₂₆H₂₆Cl₂FeN₄: 521.26

### EXAMPLE 6

### FeCl(C₁₆H₁₁N₄)

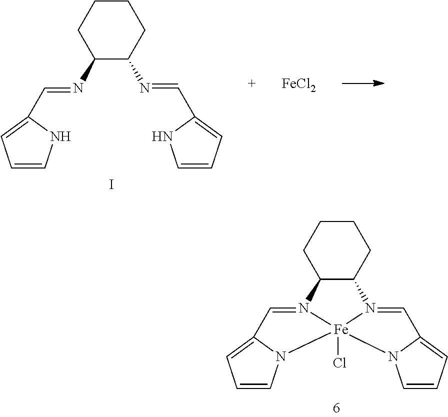

To a solution of 0.1 g of ligand l) (0.38 mmol, 1.0 equiv.) in 10 ml of CH₂Cl₂ was added 0.062 g of FeCl₃ (0.38 mmol, 1 equiv.). After stirring for 24 hours, the dark blue solution was filtered under protective gas and the solvent was removed.

Yield: 0.052 g of light brown crystals (0.014 mmol, 50%).

¹H/¹³C-NMR: NMR spectra were not possible due to the paramagnetism of Fe(III).

### EXAMPLE 7

### CoCl(C₁₆H₁₁N₄)

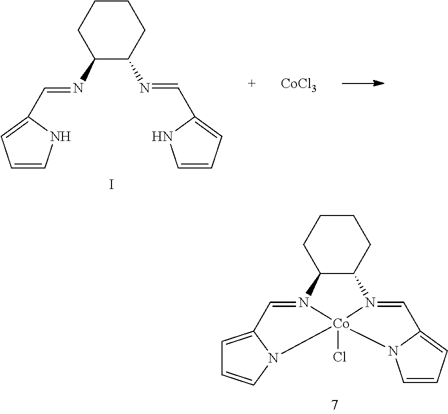

To a solution of 0.1 g of ligand l (0.38 mmol, 1.0 equiv.) in 10 ml of dry MeOH was added 0.067 g of CoCl₃ (0.38 mmol, 1 equiv.). After stirring for 24 hours, the dark blue solution changed color to reddish and was filtered under protective gas, and the solvent was removed. 0.033 g of LiCl (0.76 mmol, 42.83 g/mol) was added and the mixture was stirred in air atmosphere for two days.

Yield: 0.049 g of dark blue crystals (0.014 mmol, 50%).

### PROCESS EXAMPLES

### Polymerization

A baked-out, protective gas-flooded 100 ml autoclave with magnetic stirrer was initially charged with approx. 10-40 mg of the iron catalyst. The appropriate amount of propylene oxide (1-20 ml) was added, which corresponds to a propylene oxide (PO)/catalyst ratio of 1:100 to 1:1000. Subsequently, CO₂ was injected (pressures of 15-21 bar). The reactor was heated to 80-100° C. and left at the temperature for between 30 min and 24 hours (variation of the reaction time). In the event of a pressure drop during the polymerization time, it was generally possible to restore the appropriate starting pressure by injection.

### Workup of the Reaction Solution

The workup was effected according to WO 03/029325. The reactor was vented and the reactor contents were poured, for example, into methanol which had been acidified with 5 ml of conc. hydrochloric acid (37% by weight). A polymer precipitated out, and was filtered off and dried under reduced pressure at 60° C.-80° C. overnight.

A further means of polymer workup is as follows: the polymer is washed quantitatively out of the reactor with, for example, dichloromethane, acetone or ethyl acetate, and then the solvent is drawn off and the product is dried.

### Process Example 1

A baked-out, protective gas-flooded 100 ml autoclave with a magnetic stirrer was initially charged with 30 mg of the iron catalyst 2.5 ml of propylene oxide (PO) were added, which corresponded to a PO/catalyst ratio of 1:1000. Subsequently, 20 bar of CO₂ were injected. The reactor was heated to 80° C. and left at the temperature for 2 hours. After 2 hours, the reactor was cooled to approx. 0° C. and vented, and the reaction mixture was withdrawn. 0.128 g of cyclic carbonate was obtained, which corresponded to a turnover frequency (TOF) of 22 h⁻¹.

### Process Example 2

A baked-out, protective gas-flooded 100 ml autoclave with a magnetic stirrer was initially charged with 30 mg of the iron catalyst 1 and 23 mg of tetrabutylammonium bromide TBAB as a cocatalyst, which corresponded to a catalyst/cocatalyst ratio of 1:2.5 ml of propylene oxide (PO) were added, which corresponded to a PO/catalyst ratio of 1:1000. Subsequently, 20 bar of CO₂ were injected. The reactor was heated to 80° C. and left at the temperature for 2 hours. After 2 hours, the reactor was cooled to approx. 0° C. and vented, and the reaction mixture was withdrawn. 5.742 g of cyclic carbonate were obtained, which corresponded to a turnover frequency (TOF) of 394 h⁻¹.

### Process Example 3

A baked-out, protective gas-flooded 100 ml autoclave with a magnetic stirrer was initially charged with 26 mg of the iron catalyst 1 and 8 mg of TBAB as a cocatalyst. 1.5 ml of cyclohexene oxide (CHO) were added, which corresponded to a CHO/catalyst ratio of 1:400. Subsequently, 20 bar of CO₂ were injected. The reactor was heated to 80° C. and left at the temperature for 2 hours. After 2 hours, the reactor was cooled to approx. 0° C. and vented, and the reaction mixture was withdrawn. 0.237 g of product with approx. 50% polycyclohexene carbonate content was obtained, which corresponded to a turnover frequency (TOF) of 14 h⁻¹.

### Process Example 4

A baked-out, protective gas-flooded 100 ml autoclave with a magnetic stirrer was initially charged with 16 mg of the iron catalyst 3 and 4.7 mg of TBAB as a cocatalyst. 1.2 ml of cyclohexene oxide were added, which corresponded to a CHO/catalyst ratio of 1:400. Subsequently, 20 bar of CO₂ were injected. The reactor was heated to 80° C. and left at the temperature for 2 hours. After 2 hours, the reactor was cooled to approx. 0° C. and vented, and the reaction mixture was withdrawn. 0.053 g of product with approx. 50% polycyclohexene carbonate content was obtained, which corresponded to a turnover frequency (TOF) of 8 h⁻¹.

The summary of further experiments can be taken from the table below. All experiments were conducted as described in process examples 1 to 4.

Experiments with propylene oxide:

| | | | TOF | cPC | PPC | PPO |
|---|---|---|---|---|---|---|
| Cat | TBAB | PO/cat | (h⁻¹) | (%) | (%) | (%) |
| 1 | 1:1 | 1000 | 394 | 100 | 0 | 0 |
| 2 | / | 1000 | 22 | 100 | 0 | 0 |
| 2 | 1:2 | 1000 | 191 | 100 | 0 | 0 |
| 3 | 1:2 | 1000 | 106 | 100 | 0 | 0 |
| 4 | 1:2 | 1000 | 163 | 95 | 0 | 5 |
| Comp. 5 | 1:1 | 100 | 382 | 100 | 0 | 0 |
| 6 | 1:2 | 400 | 13 | 100 | 0 | 0 |

Experiments with cyclohexene oxide:

| | | | TOF | cCHC | PCHC | PCHO |
|---|---|---|---|---|---|---|
| Cat | TBAB | CHO/cat | (h⁻¹) | (%) | (%) | (%) |
| 1 | 1:2 | 400 | 14 | 0 | 50 | 50 |
| 3 | 1:2 | 400 | 8 | 0 | 50 | 50 |
| Comp. 5 | / | 1000 | 4 | 0 | 18 | 18 |

cPC: cyclic propylene carbonate
PPC: polypropylene carbonate
PPO: polypropylene oxide
cCHC: cyclic cyclohexene carbonate
PCHC: polycyclohexene carbonate
PCHO: polycyclohexene oxide

1-6. (canceled)

7. A process for preparing carbonates which comprises reacting propylene oxide, ethylene oxide, styrene oxide and/or cyclohexene oxide with carbon dioxide in the presence of one or more catalysts of the formula I 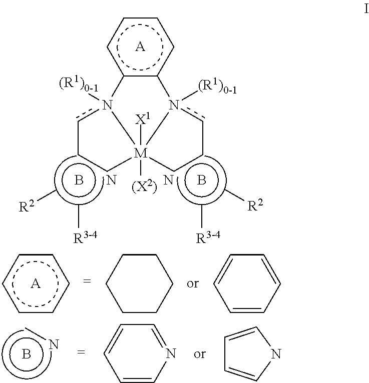
where
R¹ is hydrogen, C₁-C₆-alkyl, C₁-C₆-haloalkyl or NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano;
R³ and R⁴ are each hydrogen or together are a butadienylene moiety which bears the R⁵ substituent;
R⁵ is C₁-C₄-alkyl, C₁-C₄-haloalkyl, halogen, amino, nitro, C₁-C₆-alkoxy or cyano;
M is Zn(II), Mg(II), Cr(II), Cr(III), Co(II), Co(III), Fe(II) or Fe(III); and
X¹ or X² are each OCOCH₃, OCOCF₃, OSO₂C₇H₇ or halogen.

8. The process according to claim 7, wherein R¹ is hydrogen or NR′₄—(CH₂)₂₋₆—and R′ is C₁-C₆-alkyl.

9. The process according to claim 7, wherein R² is methyl, tert-butyl, trifluoromethyl, fluorine, chlorine, bromine, iodine, methoxy, trifluoromethoxy or cyano.

10. A catalyst of the formula Ia 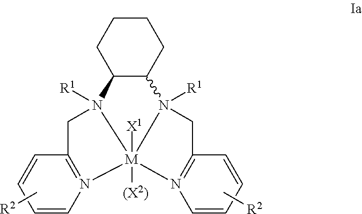
where
R¹ is hydrogen, C₁-C₆-alkyl, NR′₄—(CH₂)₂₋₆—where R′ is C₁-C₆-alkyl;
R² is hydrogen, C₁-C₄-alkyl, C₁-C₄-haloalkyl, halogen, amino, nitro, C₁-C₆-alkoxy or cyano;
M is Zn(II), Co(II), Co(III), Fe(II) or Fe(III); and
X¹ and X² are each independently OCOCH₃, OCOCF₃, OSO₂C₇H₇ or halogen.

11. A catalyst of the formula Ib 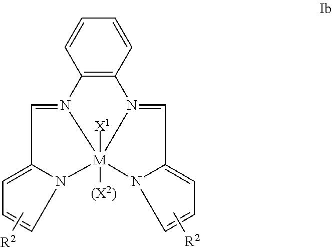
where
R² is hydrogen, C₁-C₄-alkyl, C₁-C₄-haloalkyl, halogen, amino, nitro, C₁-C₆-alkoxy or cyano;
M is Zn(II), Co(II), Co(III), Fe(II) or Fe(III); and
X¹ is OCOCH₃, OCOCF₃, OSO₂C₇H₇ or halogen.

12. A process for preparing polycyclohexylene carbonates which comprises reacting cyclohexene oxide with carbon dioxide in the presence of one or more catalysts according to claim 10.
